(19) United States
(12) Patent Application Publication
(10) Pub. No.: US 2022/0087789 A1
(43) Pub. Date: Mar. 24, 2022

(54) Method of Creating a Dental Implant Drill-Guide

(71) Applicant: Adam Brian Nulty, Bury (GB)

(72) Inventor: Adam Brian Nulty, Bury (GB)

(21) Appl. No.: 17/541,212

(22) Filed: Dec. 2, 2021

Related U.S. Application Data
(63) Continuation-in-part of application No. 16/076,320, filed on Aug. 7, 2018.

(30) Foreign Application Priority Data
Feb. 13, 2016 (GB) 1602598.3
Aug. 15, 2016 (GB) 1613924.8

(51) Int. Cl. A61C 8/00; A61C 1/08; A61B 6/14; A61B 6/03; A61B 6/00

(52) U.S. Cl. CPC A61C 8/0089; A61C 1/084; A61B 6/4085; A61B 6/032; A61B 6/14

(57) ABSTRACT

A method of creating a dental implant drill-guide is provided comprising the steps of using mucosal impression data of an edentulous maxillary or mandibular arch having a plurality of orthodontic screws inserted into the osseous tissue thereof and which extend through the gingival mucosa, along with image data of the arch to determine dental implant position data for the insertion of one or more dental implants into the edentulous maxillary or mandibular arch relative to the positions of the plurality of orthodontic screws. It is then possible to create a dental implant drill-guide to guide the drilling of dental-implant cavities in the edentulous maxillary or mandibular arch, and subsequently create a dental implant drill-guide from the dental implant drill-guide parameter data, the dental implant drill-guide having a plurality of orthodontic screw-seats which are engagable with the said plurality of orthodontic screws.

### CROSS REFERENCE TO RELATED APPLICATIONS

The present application is a Continuation-In-Part Application of U.S. patent application Ser. No. 16/076,320 filed on Aug. 7, 2018, which is a national phase application of PCT Application No. PCT/GB2017/050251 filed on Feb. 2, 2017, which claims the benefit of UK Patent Application Nos. 1613924.8 filed on Aug. 15, 2016 and 1602598.3 filed on Feb. 13, 2016. All the above are hereby incorporated by reference in their entirety.

The present invention relates to a method of creating a dental implant drill-guide, in particular for the drilling of dental implant cavities in an edentulous maxillary or mandibular arch of a patient. The invention further relates to a further method of creating a dental implant drill-guide, and further to a method of implanting one or more dental implants into a patient.

Dental implants are artificial replacements for missing teeth which interface with the bone of the maxilla or mandible. Often the term dental implant refers solely to the bone-interfacing element, which is often screw threaded, to which a false crown of a tooth is mounted.

Ordinarily, if only a single tooth or a few teeth are missing, then the dental surgeon is able to align the dental implant to the remaining teeth. However, where a patient is fully edentulous, that is, missing all teeth emanating from the mandible or maxilla, then there is a difficulty in referencing the positions in which the dental implants should be inserted.

One option is to cut through the gingival mucosa or gums to expose the underlying bone structure, allowing the dental surgeon to determine visually where to drill to insert the dental implant. This is highly traumatic to the patient, and can take a significant amount of time for the mucosa to heal.

An alternative is to take a mucosal impression using an impression body, imaging the osseous tissue underneath the gingival mucosa, and then applying a drill guide over the mucosa to align the dental drill bit. The problem with this approach is that when the mucosal impression is taken, the gingival mucosa deform or compress under the force applied, skewing the information available to the dental surgeon. This can result in misaligned dental implants, even where a drill guide is used.

The present invention seeks to provide a method of forming a drill guide which is more accurate than that currently available, without significantly increasing the need for invasive surgery at or adjacent to the gums of the patient.

According to a first aspect of the invention, there is provided a method of creating a dental implant drill-guide for guiding the drilling of dental-implant cavities in an edentulous maxillary or mandibular arch, the method comprising the steps of: a] using mucosal impression data of an edentulous maxillary or mandibular arch having a plurality of orthodontic screws inserted into the osseous tissue thereof and which extend through the gingival mucosa, along with image data of the edentulous maxillary or mandibular arch to determine dental implant position data for the insertion of one or more dental implants into the edentulous maxillary or mandibular arch relative to the positions of the plurality of orthodontic screws; b] forming dental implant drill-guide parameter data from the determined dental implant position data; and c] creating a dental implant drill-guide from the determined dental implant drill-guide parameter data, the dental implant drill-guide having a plurality of orthodontic screw-seats which correspond to the said plurality of orthodontic screws of the mucosal impression data.

By relying on data relating to orthodontic screws inserted into the maxillary or mandibular arch prior to imaging the jaw, only a small amount of penetration of the gingival mucosa is required. However, the orthodontic screws are able to anchor into the mandible or maxilla of the patient, providing an accurate and reproducible reference frame for the operator when manufacturing a bespoke drill guide for the patient. This ensures that the drill guide is correctly aligned with respect to the bone, rather than the mucosal tissue, which is compressible and mobile.

Preferably, during step a], the mucosal impression data may be determined using three said orthodontic screws which are inserted in a triangular arrangement in the edentulous maxillary or mandibular arch. Optionally, during step a], the mucosal impression data may be determined using the plurality of orthodontic screws which are inserted between 2 and 3 millimetres deep into the osseous tissue of the edentulous maxillary or mandibular arch.

During step a], a digital mucosal impression of the edentulous maxillary or mandibular arch may be obtained.

A triangulated arrangement of orthodontic screws has the advantage of providing sufficient structural support to the dental implant drill guide after seating onto the screws, whilst minimising the need to invasively insert the screws through the gingival mucosa of the patient. Minimisation of the pain caused to the patient is therefore of prime concern.

The method may further comprise a step prior to step a] of obtaining pre-operative imaging data of the edentulous maxillary or mandibular arch to determine an optimal position for the plurality of orthodontic screws and/or an optimal position of the dental-implant cavities. Said pre-operative imaging data is radiographic imaging data. Preferably, during step a], the imaging data may be Cone Beam Computed Tomography imaging data.

By imaging the bone structure of the patient prior to insertion of the orthodontic screws, the major nerves can be readily avoided, and a most effective arrangement of dental implant cavities with respect to orthodontic screw positions can be ascertained.

Optionally, during step c], the method may further comprise the step of engaging the orthodontic screw seats of the dental implant with the plurality of orthodontic screws using a positive locator. The positive locators may preferably include a collar of each orthodontic screw which is receivable by the dental implant drill-guide

The better the engagement between the drill guide and the orthodontic screws, the simpler it will be to secure the drill guide into position, advantageously simplifying the installation process for the dental surgeon.

Preferably, the apical part of the two-part orthodontic screw is a non-integrating orthodontic screw, the apical part of the two-part orthodontic screw acts as a positional reference for the dental implant drill-guide.

Optionally, during step a] the mucosal impression data is refined using pre-existing characteristic shape data corresponding to the two-part orthodontic screws. This may allow for better engagement and alignment between the drill guide and the orthodontic screws, as the two-part orthodontic screws will be more accurately imaged. Advantageously, this will simplify the installation process.

According to the invention, there is provided a dental implant drill-guide system comprising: a plurality of two-part orthodontic screws, a coronal part of each two-part orthodontic screw having a screw-thread which is receivable within an apical part of the two-part orthodontic screw; and a dental implant drill-guide, the dental implant drill-guide having a guide body which is complementarily-shaped to an edentulous maxillary or mandibular arch of a patient, a plurality of first apertures through the guide body each having an orthodontic screw-seat associated therewith which is engagable with the apical part of one said two-part orthodontic screw, and at least one second aperture which is oriented and sized to form a drill-guide through which a dental drill for drilling a dental-implant cavity in the edentulous maxillary or mandibular arch can pass.

The provision of the two-part orthodontic screws allows for a simple and effective means of clamping a dental guide in position, whilst the orthodontic screw seats on the dental implant drill guide advantageously simplifies the insertion and engagement of the drill guide in situ.

Preferably, the or each second aperture may be larger than the or each first aperture of the dental implant drill-guide. Furthermore, the system may further comprise a positive locator between each orthodontic screw-seat and a head of the apical part of one said two-part orthodontic screw. The positive locator may optionally include a collar of the apical part of the two-part orthodontic screw which is receivable by a respective first aperture of the dental implant drill guide

Not only can the size of the apertures be made so as to minimise spatial constraints whilst providing sufficient drilling and screwing room, but this also acts as a straightforward visual guide for a dental surgeon to assist with alignment and fixation of the drill guide into position.

Optionally, the guide body may comprise an anterior frame element, a posterior frame element, and at least one bridging elements, the first and second apertures being located within the or each bridging element, and/or at least one buccal abutment element.

The guide body is beneficially formed so as to be readily receivable about the edentulous maxillary or mandibular arch of a patient, whilst also providing ample space for a dental surgeon to be able to operate within the restricted oral cavity.

Preferably, the coronal part of each two-part orthodontic screw may be held captive within each respective first aperture.

By holding the coronal part of each orthodontic screw with respect to the guide body, the dental surgeon can safely manipulate and engage the screw without there being a risk of the coronal part of the screw becoming dislodged and entering into the patient's oesophagus or trachea. Furthermore, if held captive within the drill-guide, the risk of losing the orthodontic screws in transit is also minimised.

According to the invention, there is provided a method of creating a dental implant drill-guide, the method comprising the steps of: a] using three-dimensional oral information data of an edentulous maxillary or mandibular arch having a plurality of orthodontic screws inserted into the osseous tissue thereof and which extend through the gingival mucosa to determine dental implant position data for the insertion of one or more dental implants into the edentulous maxillary or mandibular arch relative to the positions of the plurality of orthodontic screws; b] forming dental implant drill-guide parameter data from the determined dental implant position data; and c] creating a dental implant drill-guide from the determined dental implant drill-guide parameter data, the dental implant drill-guide having a plurality of orthodontic screw-seats which correspond to the said plurality of orthodontic screws of the three-dimensional impression data.

It will be apparent that whilst current techniques for determining oral geometry require a two-stage information gathering process, that is, the imaging of the jaw and the taking of an oral impression, it may be possible to fully reconstruct the oral geometry in a single stage, in which case, the present methodology will still be applicable.

According to the invention, there is provided a method of assembling a drill guide for the insertion of dental implants, the method comprising the steps of: a] providing a dental implant drill-guide system, preferably in accordance with the third aspect of the invention; b] engaging the orthodontic screw-seats of the dental implant drill-guide respectively with the apical parts of the two-part orthodontic screws; and c] fixing the dental implant drill-guide in position by engaging the coronal parts of the two-part orthodontic screws with the apical parts of the two-part orthodontic screws so as to clamp the dental implant drill-guide into position.

According to the invention, there is provided a method of creating a dental implant drill-guide for guiding the drilling of dental-implant cavities in an edentulous maxillary or mandibular arch, the method comprising the steps of: a] inserting a plurality of apical parts of two-part orthodontic screws into an edentulous area of a maxillary or mandibular arch of a patient, such that the two-part orthodontic screws engage with the osseous tissue of the edentulous area of the maxillary or mandibular arch and extend through the gingival mucosa; b] obtaining impression data of the edentulous area of the maxillary or mandibular arch inclusive of the plurality of two-part orthodontic screws to form a maxillary or mandibular image; c] refining the impression data based on characteristic shape data corresponding to the two-part orthodontic screws; d] determining dental implant position data for the insertion of one or more dental implants into the edentulous area of the maxillary or mandibular arch relative to the positions of the plurality of two-part orthodontic screws from the refined impression data; and e] creating a dental implant drill-guide from the determined dental implant position data, the dental implant drill-guide having a plurality of orthodontic screw-seats which correspond to the said plurality of two-part orthodontic screws.

Using data relating to orthodontic screws inserted into the maxillary or mandibular arch prior to imaging the jaw, requires only a small amount of penetration of the gingival mucosa. The orthodontic screws are able to anchor into the mandible or maxilla of the patient, providing an accurate and reproducible reference frame for the operator when manufacturing a bespoke drill guide for the patient. This ensures that the drill guide is correctly aligned with respect to the bone, rather than the mucosal tissue, which is compressible and mobile. The refinement of the impression data using characteristic shape data corresponding to the two-part orthodontic screws is advantageous as it allows for better engagement and alignment between the drill guide and the orthodontic screws, as the two-part orthodontic screws will be more accurately imaged. As such, the installation process for the dental surgeon will be simplified.

Preferably, the coronal part of each of the two-part orthodontic screws, has a screw-thread which is receivable within an apical part of the two-part orthodontic screw. The characteristic shape data may relate to a shape of the two-part orthodontic screw which is: pyramidal frustum; substantially pyramidal frustum; conical frustum; or substantially conical frustum.

Using two-part orthodontic screws which have a shape of pyramidal frustum; substantially pyramidal frustum; conical frustum; or substantially conical frustum allows for more accurate imaging of the two-part orthodontic screws as the shapes are easier to refine.

Preferably, during step a], the plurality of orthodontic screws is inserted in a triangular arrangement in the edentulous area of the maxillary or mandibular arch. Optionally, during step a], the plurality of orthodontic screws is inserted between 2 and 3 millimetres deep into the osseous tissue of the edentulous maxillary or mandibular arch.

A triangulated arrangement of orthodontic screws has the advantage of providing sufficient structural support to the dental implant drill guide after seating onto the screws, whilst minimising the need to invasively insert the screws through the gingival mucosa of the patient. Minimisation of the pain caused to the patient is therefore of prime concern.

The method may further comprise a step prior to step a] of obtaining pre-operative imaging data of the edentulous maxillary or mandibular arch to determine an optimal position for the plurality of orthodontic screws and/or an optimal position of the dental-implant cavities. Said pre-operative imaging data is radiographic imaging data. Preferably, during step b], image data of the edentulous area of the maxillary or mandibular arch is used alongside the impression data to form the maxillary or mandibular image.

By imaging the bone structure of the patient prior to insertion of the plurality of orthodontic screws, the major nerves can be readily avoided. Additionally, it allows for a most successful arrangement of dental implant cavities with respect to orthodontic screw positions to be determined. Use of the image data of the edentulous area of the maxillary or mandibular arch allows the impression data to be clarified.

According to the invention, there is provided a method of implanting one or more dental implants into a patient, the method comprising the steps of: a] inserting a plurality of apical parts of two-part orthodontic screws into an edentulous maxillary or mandibular arch of a patient, such that the two-part orthodontic screws engage with the osseous tissue of the edentulous maxillary or mandibular arch; b] obtaining a mucosal impression of the edentulous maxillary or mandibular arch; c] imaging the maxilla or mandible of the patient inclusive of the plurality of two-part orthodontic screws to form a maxillary or mandibular image; d] determining from the maxillary or mandibular image and the mucosal impression one or more dental implant positions for the insertion of dental implants into the edentulous maxillary or mandibular arch relative to the positions of the plurality of two-part orthodontic screws; e] creating a dental implant drill-guide based on the one or more dental implant positions and the positions of the plurality of two-part orthodontic screws; f] positioning the dental drill-guide on the apical parts of the two-part orthodontic screws; g] fixing the dental implant drill-guide in position by engaging the coronal parts of the two-part orthodontic screws with the apical parts of the two-part orthodontic screws so as to clamp the dental implant drill-guide; h] drilling one or more dental-implant cavities in the edentulous maxillary or mandibular arch using the dental implant drill-guide; and i] inserting a dental implant into each dental-implant cavity.

Preferably, during step i], the dental implants may be guided into the respective dental-implant cavities by the dental implant drill-guide

The invention will now be more particularly described, by way of example only, with reference to the accompanying drawings, in which:

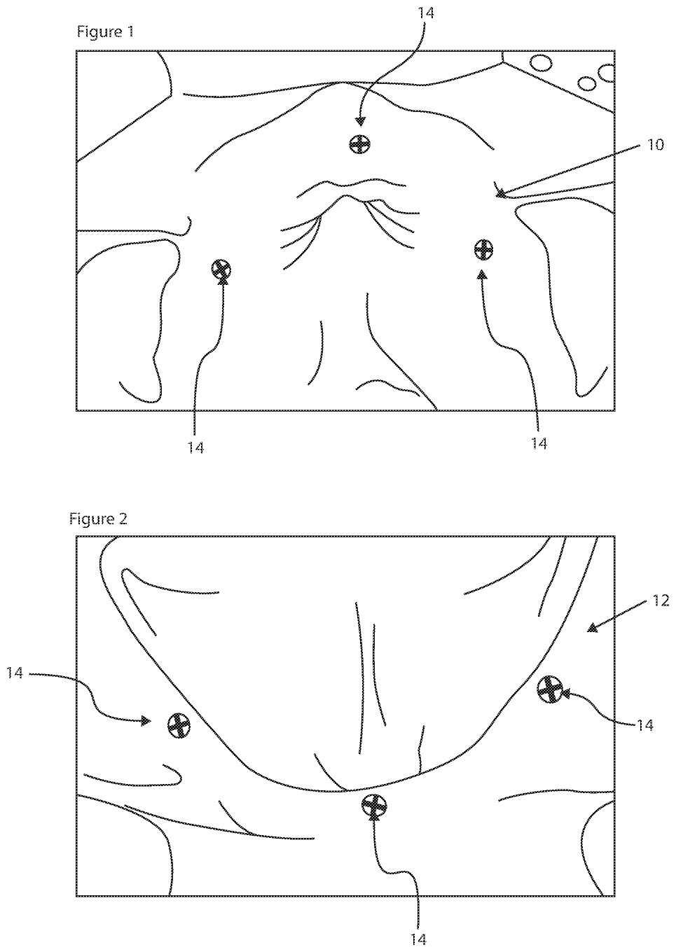

FIG. 1 is a representation of a maxillary edentulous arch of a patient having a plurality of orthodontic screws inserted therein in a triangular formation, in accordance with the present invention;

FIG. 2 is a representation of a mandibular edentulous arch of a patient having a plurality of orthodontic screws inserted therein in a triangular formation, in accordance with the present invention;

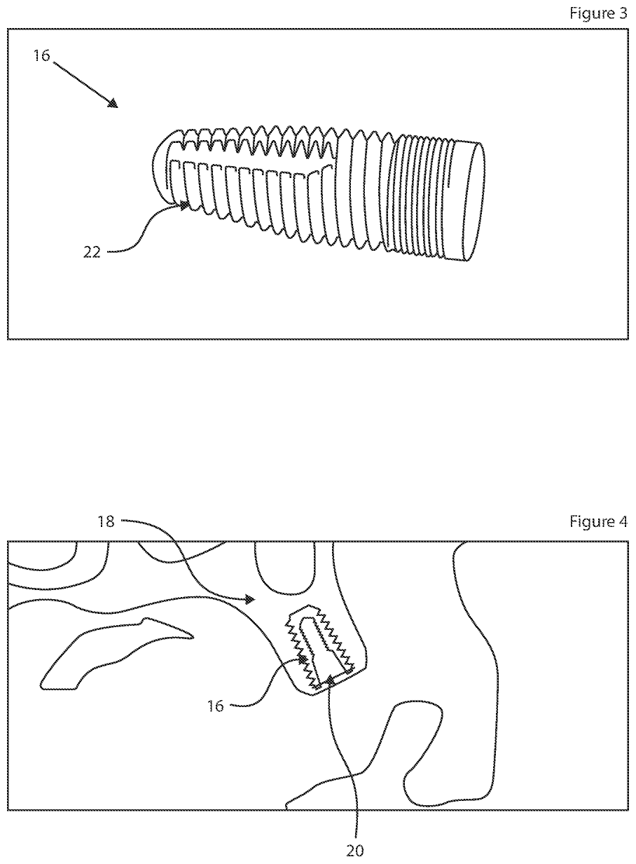

FIG. 3 is a perspective representation of a dental implant to be inserted into a maxillary or mandibular edentulous arch of a patient, in accordance with the present invention;

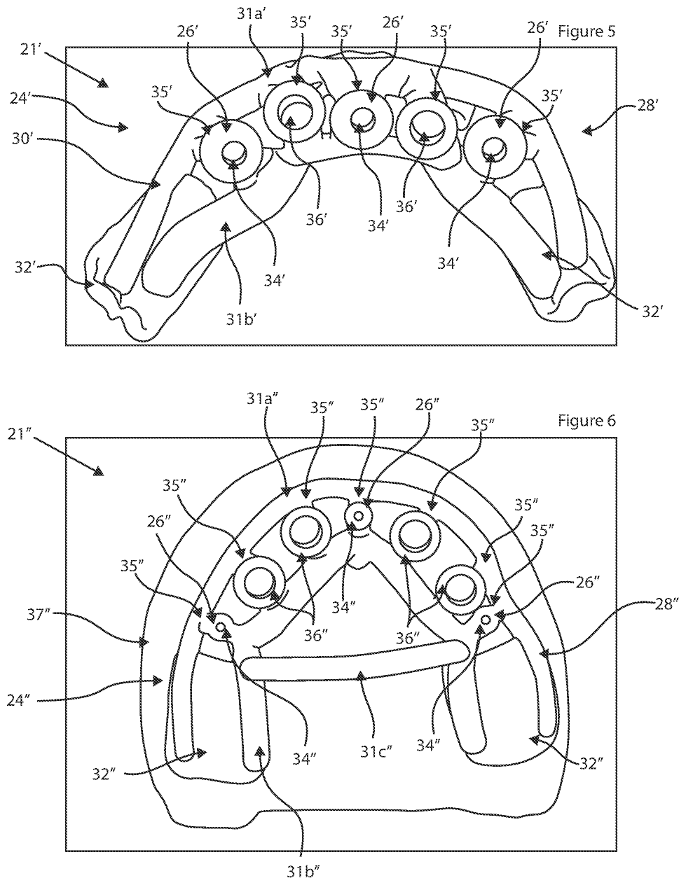

FIG. 4 is a cross-sectional image through a maxilla of a patient, indicating a viable dental-implant cavity which has been drilled, including an overlain cross-sectional representation of the dental implant of FIG. 3, in accordance with the present invention;

FIG. 5 is a plan view of a first embodiment of a digital model of a dental implant drill-guide formed in accordance with the first aspect of the invention;

FIG. 6 is a plan view of a second embodiment of a digital model of a dental implant drill-guide formed in accordance with the first aspect of the invention, interacting with a digital model of an edentulous arch of a patient;

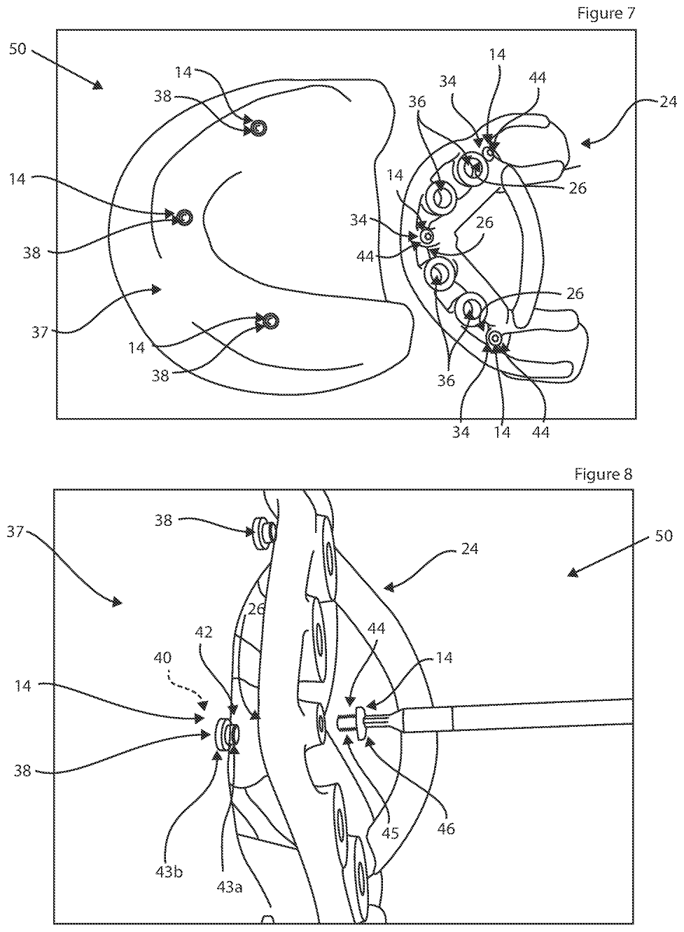

FIG. 7 is a plan view of a first embodiment of a dental implant drill-guide formed in accordance with the first aspect of the invention in combination with a dummy edentulous arch;

FIG. 8 is an enlarged perspective representation of the dental implant drill-guide being inserted into the dummy edentulous arch using a two-part orthodontic screw;

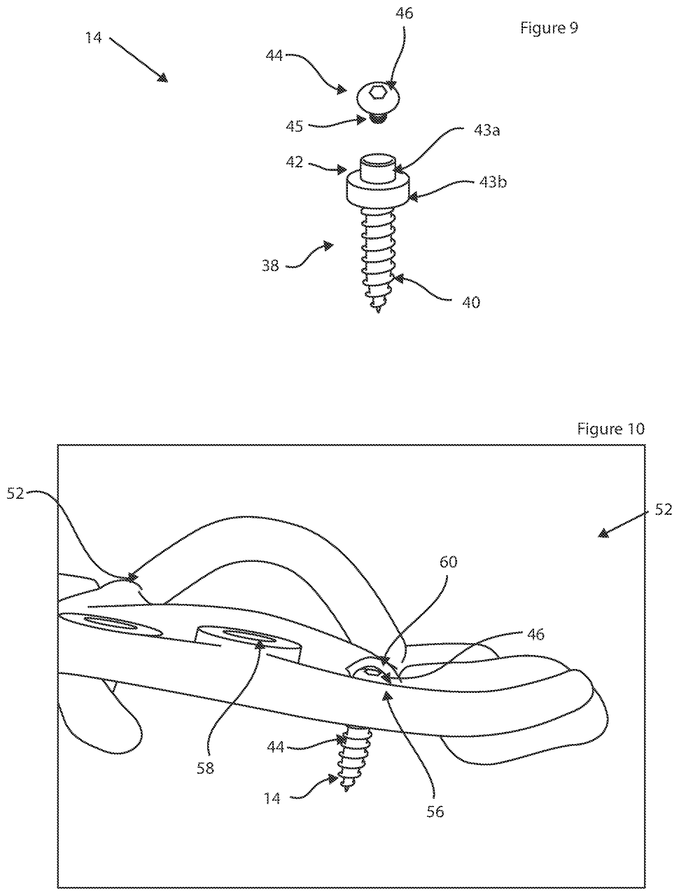

FIG. 9 is an exploded perspective representation of the two-part orthodontic screw shown in FIG. 8, in isolation from the dental implant drill-guide;

FIG. 10 is a plan view of a second embodiment of a dental implant drill-guide formed in accordance with the first aspect of the invention;

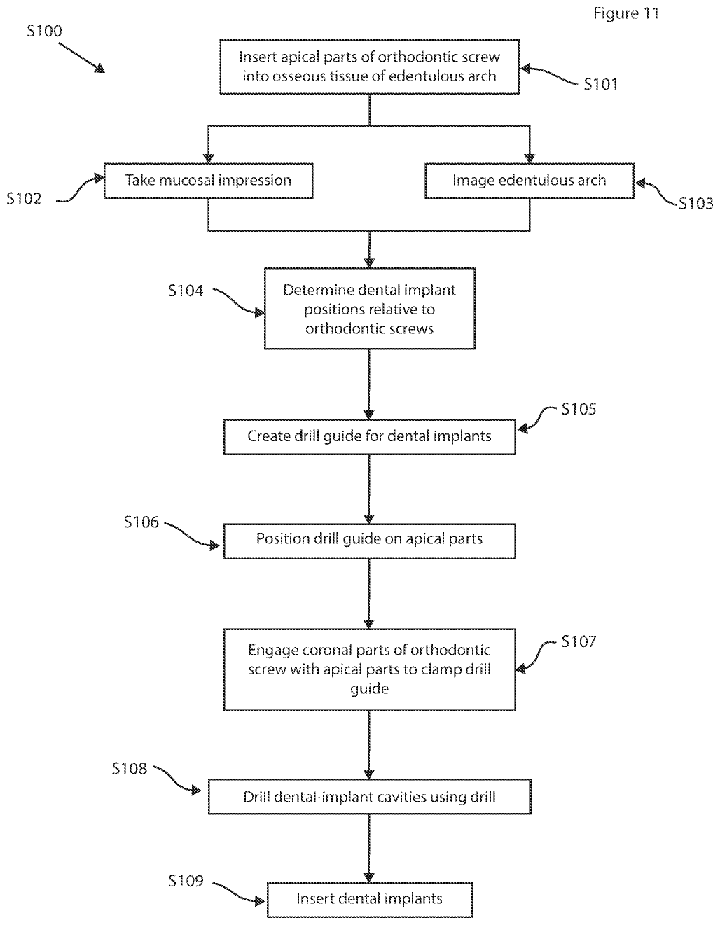

FIG. 11 is a diagrammatic representation of a method of implanting one or more dental implants into a patient, in accordance with the invention;

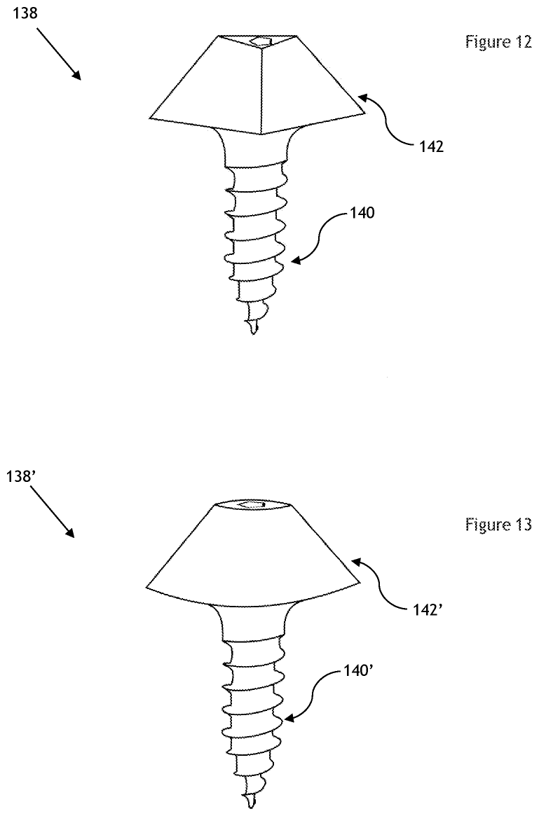

FIG. 12 is a front perspective representation of a second embodiment of an apical part of a two-part orthodontic screw for use in a method in accordance with the invention;

FIG. 13 is a front perspective representation of a third embodiment of an apical part of a two-part orthodontic screw for use in a method in accordance with the invention;

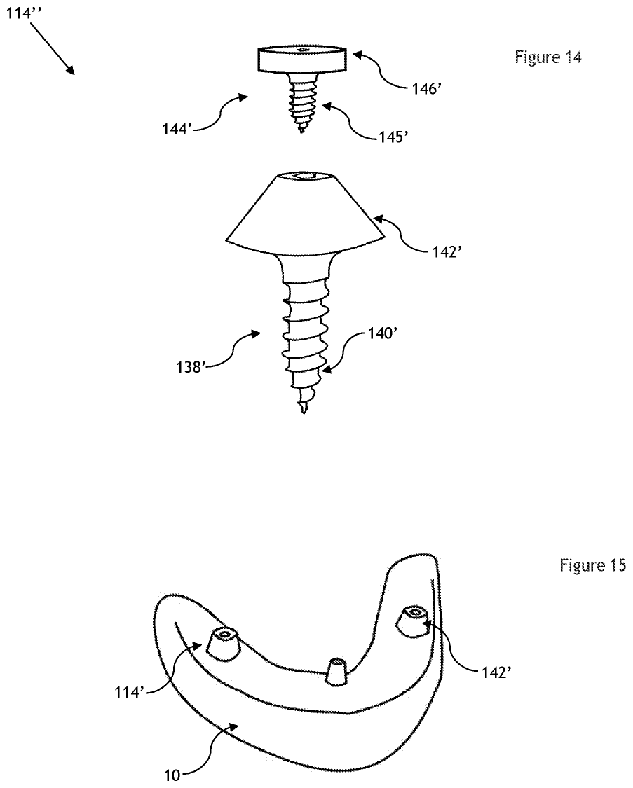

FIG. 14 is an exploded perspective representation of a third embodiment of a two-part orthodontic screw for use in a method in accordance with the invention;

FIG. 15 is a representation of an edentulous area of a mandibular arch of a patient having a plurality of two-part orthodontic screws inserted therein in a triangular formation, in accordance with invention; and

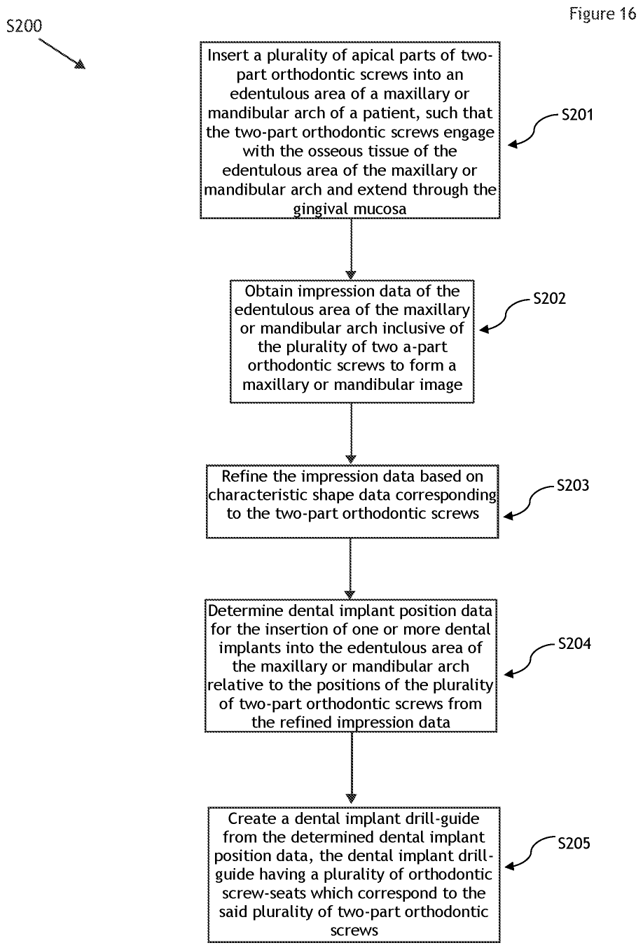

FIG. 16 is a diagrammatic representation of a method of creating a dental implant drill-guide for guiding the drilling of dental-implant cavities in an edentulous area of a maxillary or mandibular arch, in accordance with the invention.

Referring firstly to FIGS. 1 and 2, maxillary and mandibular edentulous arches 10, 12 of a patient are shown, that is the toothless arches inside the mouth of a patient, prior to the installation of one or more dental implants to replace the patient's missing teeth.

A plurality of orthodontic screws 14 are inserted into the maxillary or mandibular edentulous arch 10, 12, and these will act as guides to the determination of dental implant positions. Preferably, three such orthodontic screws 14 are provided in a triangular formation so as to provide sufficient reference points in the maxillary or mandibular edentulous arch 10, 12 whilst creating minimal invasion into the patient's mouth.

It may be possible to pre-operatively image the maxillary or mandibular edentulous arch 10, 12 prior to insertion of the orthodontic screws 14 in order to not only determine an optimal position of each orthodontic screw 14, but also an optimal position of the dental implants to be installed. This may be achieved by radiographic imaging of the maxilla or mandible to determine a position of the osseous tissue.

It is noted that the term orthodontic screw 14 is preferably intended to refer to a screw which can be inserted into the maxillary or mandibular edentulous arch 10, 12 of a patient, preferably so as to engage with the osseous tissue by no more than around 2 or 3 millimetres. This prevents the orthodontic screw 14 from integrating with the osseous tissue. On the other hand, a dental implant 16, such as that illustrated in FIG. 3, is preferably designed to integrate with the osseous tissue 18 within a dental-implant cavity 20, such as that illustrated in FIG. 4, and therefore the screw thread 22 of the dental implant 16 penetrates much more deeply into the osseous tissue 18 so as to form a more permanent bond in the patient's mouth.

Once an image of the maxillary or mandibular edentulous arch 10, 12 has been taken, the optimum positions of the orthodontic screws 14 and/or the dental-implant cavities 20 may be determined. Various factors may affect where the said optimum positions may be, for example, the intended positions of the dental implants 16, the underlying structure of the osseous tissue, the positions of nerve endings within the maxillary or mandibular edentulous arch 10, 12, and/or the angular positioning of the maxillary or mandibular edentulous arch 10, 12. This may typically be assessed by converting the image of the maxillary or mandibular edentulous arch 10, 12 into pre-operative imaging data which can be processed to determine the respective optimum positions of the orthodontic screws 14 and dental-implant cavities 20.

Once the dentist has inserted the plurality of orthodontic screws 14 into the maxillary or mandibular edentulous arch 10, 12 of the patient, it is possible to obtain mucosal impression data relating to the gingival mucosa of the patient. This may preferably be achieved by inserting an impression body into the mouth of the patient, which may be preferably formed from a mucostatic or mucocompressive material, about which the patient may clamp their jaws. This forms an imprint of the patient's gingival mucosa onto the impression body inclusive of an imprint of the heads of the orthodontic screws 14.

The orthodontic screws 14 therefore act to provide a rigid support for the impression body which is fixed relative to the osseous tissue 18 of the maxillary or mandibular edentulous arch 10, 12. Ordinarily, the gingival mucosa of an edentulous patient will compress during the creation of the impression, which would lead to an inaccurate impression relative to the osseous tissue. The impression may then be converted into mucosal impression data, preferably digital mucosal impression data, to provide three-dimensional information regarding the inside of the patient's mouth.

In addition to collating mucosal impression data, it is also possible to image the maxillary or mandibular edentulous arch 10, 12, preferably using a Cone Beam Computer Tomography (CBCT) scanning device to obtain a CBCT scan. This will yield imaging data relating to the maxillary or mandibular edentulous arch 10, 12 below the surface of the gingival mucosa in three dimensions, and will also image the orthodontic screws 14 to provide the necessary imaging data to calculate the drilling angles and positions in order to create the dental-implant cavities 20.

By combining the mucosal impression data with the imaging data of the maxillary or mandibular edentulous arch 10, 12, it is possible to determine dental implant position data for the insertion of one or more dental implants 16 into the edentulous maxillary or mandibular arch 10, 12 relative to the positions of the plurality of orthodontic screws 14. This may be achieved using a computer software matching program, for instance.

It will be appreciated that medical advances may progress such that the imaging of the patient's oral cavity and underlying maxilla and mandible can be achieved with a single scan, and therefore separate mucosal impression and CBCT scanning may not strictly be required. Furthermore, whilst the mucosal impression is presently performed prior to the CBCT scan, there is no strict requirement for this to be the case, and the order could be reversed.

Once the dental implant position data has been created, it may be possible for dental implant drill-guide parameter data to be ascertained, preferably using computer software, which creates a digital model of a dental implant drill guide, such as the digital model 24′ shown in FIG. 5, having areas which are capable of guiding a dental drill into the mouth of the patient to create correctly positioned and oriented dental-implant cavities 20.

This digital model 21′ is indicative of a physical dental implant drill-guide 24′ having a plurality of orthodontic screw-seats 26′ which are sized and shaped to be engagable with the said plurality of orthodontic screws 14. The dental implant drill-guide 24′ is designed for at least the guiding of a drill in the oral cavity of a patient, and preferably also for the subsequent guidance of dental implants into the implant cavities formed by the drill.

The digital model 21′ of the dental implant drill-guide 24′ has a guide body 28′ which is sized and shaped to be receivably insertable into the mouth of an edentulous patient at or adjacent to the maxillary or mandibular edentulous arch 10, 12. The representation of the guide body 28′ here comprises an arched frame 30′ which matches or substantially matches the curvature of the maxillary or mandibular edentulous arch 10, 12, which may be formed having anterior and posterior frame elements 31a, 31b. These anterior and posterior frame elements 31a′, 31b′ may be formed having unequal dimensions, which may assist with the comfortable seating of the dental implant drill-guide 24 in the patient's oral cavity. The arched frame 30′ may terminate at each end in buccal abutment elements 32′ which are seatable at or adjacent to the buccae of the edentulous patient. It will be appreciated, however, that the guide body 28′ may be formed as a single contiguous element, into which apertures can be directly formed.

In the arched frame 30′, a plurality of apertures may preferably be provided, which may be distinguishable as a set of first apertures 34′ which are associable with the orthodontic screws 14 and a set of second apertures 36′ which are associable with the dental implants 16. In the depicted embodiment of dental implant drill-guide 24′, there are three first apertures 34′, which may be held in place by bridging elements 35′, and two second apertures 36′, this being a digital model 21′ of a dental implant drill-guide 24′ for the installation of two dental implants 16 into the edentulous patient. Each of the second apertures 34′, 36′ may be also formed through bridging elements 35′ preferably interposed between the anterior and posterior frame elements 31a′, 31b′.

Here, the first apertures 34′ are formed so as to each include an orthodontic screw-seat 26′ which is engagable with, preferably the head of, an orthodontic screw 14 in the maxillary or mandibular edentulous arch 10, 12. This orthodontic screw-seat 26′ may be formed as a flat surface of a bridging element 35′ which is restable upon the heads of the orthodontic screws 14 when the dental implant drill-guide 24′ is inserted into the mouth of a patient. However, the orthodontic screw-seat 26′ may be formed so as to have a recess therein, or similar positive locator, which is engagable with the head of the orthodontic screw 14 to ensure a more secure positioning of the dental implant drill-guide 24′ on the orthodontic screws 14.

The second apertures 36′ are sized such that an appropriate dental drill-bit can pass through the second aperture 36′ in a correct alignment so as to create the correctly positioned and oriented dental-implant cavities 20′. Typically, this will mean that the second apertures 36 have a greater width or diameter than the first apertures 34′. It will be appreciated that the second apertures 36′ may not all be of a consistent size however; the second apertures 36′ may be sized appropriately for a drill bit and/or dental implant 16 to pass therethrough, which may be different depending upon the size of dental-implant cavity 20 required.

The first apertures 34′ may be sized so as to be sufficiently wide so as to receive a screw thread of an orthodontic screw 14 therethrough, but not the head of said orthodontic screw 14.

The bridging elements 35′ may be specifically sized and aligned to readily guide a drilling direction. As can be seen in FIG. 5, the angular alignment of the second apertures 36′ may be skewed and/or non-parallel. This advantageously ensures that the most beneficial arrangement of the dental implants can be produced using a single dental implant drill-guide 24′.

A second embodiment of a digital model 21″ of dental implant drill-guide 24″ is shown in FIG. 6. This embodiment is illustrated in conjunction with a digital model of a dummy edentulous arch 37″. Identical or similar components of the first embodiment of the digital model 21′ are indicated using identical or similar reference numerals, and further detailed description will be omitted for brevity.

In this embodiment, the digital model of the dummy edentulous arch 37″ has been produced based on the mucosal impression data, whereas the digital model 21″ of the dental implant drill guide 24″ has been produced based on a combination of the mucosal impression data and imaging data.

The guide body 28″ of the digital model 21″ of dental implant guide 24″ is similar to that of the first embodiment; there are anterior and posterior frame elements 31a″, 31b″ which have bridging elements 35″ therebetween supporting a plurality of first and second apertures 34″, 36″. In this embodiment, the second apertures 36″ are noticeably larger than the first apertures 34″, such that a smaller clamping area for the orthodontic screw seats 26″ is provided on the first apertures 34″. Four second apertures 36″ are provided here which are suitable for the implanting of four dental implants 20.

Instead of buccal abutment elements, the guide body 38″ here terminates instead in gingival seats 32″ which are suitable for seating onto the rear plateaus of the gingiva of a patient. A reinforcing frame element 31c″ may also be provided which bridges the left and right sides of the posterior frame element 31b″ to provide structural reinforcement of the dental implant guide 24″.

The provision of the digital model of the dummy edentulous arch 37″ and the digital model 21″ of the dental implant drill-guide 24″ allows for the exact positioning and form of the eventual physical dental implant drill-guide to be optimised for the specific patient.

Once the digital model 21″ of the dental implant drill-guide 24″ has been satisfactorily completed, a physical dental implant drill guide 24 can be produced. Such a dental implant drill-guide 24 can be seen in FIG. 7. The dental implant drill-guide 24 depicted has been formed as per the digital model 21″ shown in FIG. 6, and corresponding reference numerals will therefore be utilised to refer to corresponding components.

Typically, the dental implant drill-guide 24 will be formed from a non-toxic plastics material, and may be manufactured via moulding, extrusion, 3d printing, and/or any traditional CAD-CAM manufacturing process. Other manufacturing techniques will be apparent to the skilled person.

The dental implant drill-guide 24 is shown in conjunction with a dummy edentulous arch 37. This may be provided for training purposes only, in order to train dental surgeons in the use of the dental implant drill-guide 24.

To utilise the dental implant drill-guide 24 once manufactured, it is inserted into the oral cavity such that the orthodontic screw-seats 26 engage with the orthodontic screws 14 in position in the maxillary or mandibular edentulous arch 10, 12. In practice, the orthodontic screw in the maxillary or mandibular edentulous arch 10, 12 may only be one part of a two-part orthodontic screw 14, in this case, the apical part 38. The apical part 38 of the two-part orthodontic screw 14 has an apical screw thread 40, embedded into the dummy edentulous arch 37 in the Figures and indicated by the dashed line in FIG. 8, and an apical head 42 which engages with the corresponding orthodontic screw-seat 26 when the dental implant drill-guide 24 is inserted into the oral cavity. This can be seen in FIGS. 7 and 8 of the drawings, and the two-part orthodontic screw is illustrated in detail in FIG. 9; the apical part 38 of the two-part orthodontic screw 14 is inserted into the dummy edentulous arch 37.

The apical head 42 may be simply formed as a plateau upon which the orthodontic screw-seat 26 may sit, but will more preferably be formed having a collar 43a which is at least in part receivable within a respective first aperture 34 of the dental implant drill-guide 24, and an apical flange 43b upon which the orthodontic screw-seat 26 is abuttably restable.

Once the dental implant drill-guide 24 is correctly seated on the apical parts 38 of the two-part orthodontic screws 14, preferably by seating on the apical flange 43b with the collar 43a entering into the first aperture 34, a coronal part 44 of the two-part orthodontic screw 14 can be inserted through each respective first aperture 34 of the dental implant drill-guide 24 to secure it in place. Each coronal part 44 has a coronal screw thread 45 which is receivable through apical head 42 of the apical part 38, preferably engaging with a corresponding screw thread within the collar 43a, and a coronal head 46 which abuttably engages with a top of the first aperture 34 with which it is engaged to apply a clamping force onto the dental implant drill-guide 24. This secures the dental implant drill-guide 24 relative to the osseous tissue of the maxillary or mandibular edentulous arch 10, 12. This process is best visualised from FIG. 8.

This securing of the dental implant drill-guide 24 allows the dentist to safely drill into the maxillary or mandibular edentulous arch 10, 12 by guiding their drill bit through the respective second apertures 36 in order to form dental-implant cavities 20 in the osseous tissue of the maxillary or mandibular edentulous arch 10, 12. Once this has been performed, the two-part orthodontic screws 14 can be removed, releasing the dental implant drill-guide 24 from the maxillary or mandibular edentulous arch 10, 12. The dental implants 16 can then be inserted into their respective dental-implant cavities 20.

By using a dental implant drill-guide 24 which has been manufactured using the orthodontic screws 14 as reference points in rigid contact with the maxillary or mandibular edentulous arch 10, 12, the accuracy and reproducibility. with which dental-implant cavities 20 can be drilled is much improved. The dental-implant drill guide 24, in conjunction with the orthodontic screws 14, therefore represents an effective dental implant drill-guide system 50 which is capable of assisting with the insertion of dental implants into a patient's edentulous maxillary or mandibular arch 10, 12.

A second embodiment of a dental implant drill-guide can be seen in FIG. 10, and is indicated globally at 52. The dental implant drill-guide 52 is substantially similar to that of the first embodiment, and further detailed description will be omitted, with the differences addressed.

The guide body 54 has first and second apertures 56, 58 as in the previously-described embodiment; however, the first apertures 56 are provided with an encapsulation portion 60 which holds the coronal part 44 of the two-part orthodontic screw 14 within the first aperture 56.

In the depicted embodiment, the encapsulation portion 60 is formed as a bridge which spans the top of the first aperture 56, retaining the coronal head 46 inside the first aperture 56. Preferably, the encapsulation portion 60 is formed so as to allow for limited longitudinal movement of the coronal part 44 within the first aperture 56, to better allow the dental surgeon to manipulate the coronal part 44 when installing the orthodontic screw 14.

Other encapsulation portions could be considered, for example, a perimeter lip, shoulder or stop around the upper circumference of the first aperture 56, which may abuttably engage with the coronal head 46. This would retain the coronal head 46 in position whilst providing unfettered access for the dental surgeon to the drive of the coronal head 44 with a screwdriver or similar implement.

Furthermore, the first aperture 56 may be provided so as to have a positive engagement means with the coronal part 44 of the two-part orthodontic screw 14. For example, one or more teeth or grips may be provided which inhibit movement of the coronal part 44 in a direction away from the apical part 38, that is, only permitting movement of the coronal part 44 in a direction towards securing of the two-part orthodontic screw 14 together.

The entire process can therefore be summarised as follows, and as indicated in FIG. 11 globally at S100. A plurality of apical parts 38 of two-part orthodontic screws 14 can be inserted, at step S101, into an edentulous maxillary or mandibular arch 10, 12 of a patient, such that the two-part orthodontic screws 14 engage with the osseous tissue of the edentulous maxillary or mandibular arch 10, 12. A mucosal impression of the edentulous maxillary or mandibular arch 10, 12 can then be obtained, at step S102, and the maxilla or mandible of the patient inclusive of the plurality of two-part orthodontic screws can be imaged, at step S103, to form a maxillary or mandibular image.

One or more dental implant positions can be determined, at step S104, from the maxillary or mandibular image and the mucosal impression for the insertion of dental implants 16 into the edentulous maxillary or mandibular arch 10, 12 relative to the positions of the plurality of two-part orthodontic screws 14, which in turn allows a dental implant drill-guide 24 to be created, at step S105, based on the one or more dental implant positions and the positions of the plurality of two-part orthodontic screws 14.

The manufactured dental drill-guide 24 can then be positioned, at step S106, on the apical parts 38 of the two-part orthodontic screws 14, the dental implant drill-guide 24 being fixed, at step S107, in position by engaging the coronal parts 44 of the two-part orthodontic screws 14 with the apical parts 38 of the two-part orthodontic screws 14 so as to clamp the dental implant drill-guide 24.

One or more dental-implant cavities 20 can then be drilled, at step S108, in the edentulous maxillary or mandibular arch 10, 12 using the dental implant drill-guide 24, and a drill cylinder or collar may be used to accurately control the angle and depth of penetration of the drill into the patient's osseous tissue 18. Such a drill cylinder or collar may be receivable within the second apertures 36, thereby preventing or limiting damage to the dental implant drill-guide 24 by the drill. A dental implant 16 is then inserted, at step S109, into each dental-implant cavity 20, preferably by using a key or similar engagement tool to screw or urge the dental implant 16 into position.

In a preferred embodiment, the dental implant 16 may be inserted whilst the dental implant drill-guide 24 is still secured in position in the patient's oral cavity, since the alignments of the second apertures 36 are such that the dental implants 16 will be readily guidable into their respective dental-implant cavities 20. On the other hand, the dental implant drill-guide 24 may block or impede the insertion of the dental implants 16, and therefore it may be prudent to first remove the dental implant drill-guide 24 prior to insertion of the dental implants 16.

A second embodiment of the two-part orthodontic screws 114 is illustrated in FIG. 12. The two-part orthodontic screws 114 have an apical part 138 and a coronal part 144. The orthodontic screw inserted into the edentulous area of the maxillary or mandibular arch 10, 12 may only be one part of a two-part orthodontic screw 114, in the described embodiments it is, the apical part 138. The apical part 138 of the two-part orthodontic screw 114 has an apical screw thread 140, which implants into the edentulous arch 37 and an apical head 142 which engages with the corresponding orthodontic screw-seat 26 when the dental implant drill-guide 24 is inserted. The apical head 142 of the two-part orthodontic screw 114 shape is pyramidal frustum. The apical head 142 is receivable into the orthodontic screw-seats 26 of the dental implant drill-guide 24. The dental implant drill-guide 24 is designed to have a plurality of orthodontic screw-seats 26 which are sized and shaped to be engagable with the said plurality of two-part orthodontic screws 14, 114.

A third embodiment of the two-part orthodontic screws 114′ is illustrated in FIG. 13. As with the second embodiment, the two-part orthodontic screws 114′ have an apical part 138′ and a coronal part 144′. The orthodontic screw implanted into the edentulous area of the maxillary or mandibular arch 10, 12 may only be one part of a two-part orthodontic screw 114′, in the described embodiments it is, the apical part 138′. The apical part 138′ of the two-part orthodontic screw 114′ has an apical screw thread 140′, which inserts into the edentulous arch 37 and an apical head 142′ which engages with the corresponding orthodontic screw-seat 26 when the dental implant drill-guide 24 is inserted. The apical head 142′ of the two-part orthodontic screw 114′ shape is conical frustum. The apical head 142′ is receivable into the orthodontic screw-seats 26 of the dental implant drill-guide 24. The dental implant drill-guide 24 is designed to have a plurality of orthodontic screw-seats 26 which are sized and shaped to be engagable with the said plurality of two-part orthodontic screws 14, 114′.

As illustrated best in FIG. 14, each coronal part 144′ has a coronal screw thread 145′ which is receivable through apical head 142′ of the apical part 138′, preferably engaging with a corresponding screw thread within the apical head 142′. The coronal part 144′ has a coronal head 146 that abuttably engages with a top part of the orthodontic screw-seat 26 in which it sits to apply a clamping force onto the dental implant drill-guide 24. The coronal head 146′ is dimensioned such as to allow for the clamping force to be engaged. This advantageously allows for the clamping force on the dental implant drill-guide 24 to be distributed.

Although only the third embodiment of the two-part orthodontic screws 114′ is shown in FIG. 14, the second embodiment of the two-part orthodontic screws 114 also has a coronal part which has a coronal screw thread receivable through the apical head 142 of the apical part 138, the coronal screw thread preferably engages with a corresponding screw thread within the apical head 142. The coronal part of the two-part orthodontic screws 114 work in the same as in the first and third embodiments.

In FIG. 15, the two-part orthodontic screws 114′ are shown inserted into the edentulous area of the maxillary or mandibular arch 10, 12, to act as guides for determining the position of dental implants. Preferably, three such orthodontic screws 114′ are provided in a triangular formation so as to provide sufficient reference points in the edentulous area of the maxillary or mandibular arch 10, 12 whilst creating minimal invasion into the patient's mouth. It is noted that the term orthodontic screw 114′ is preferably intended to refer to a screw which can be inserted into the maxillary or mandibular edentulous arch 10, 12 of a patient, preferably so as to engage with the osseous tissue by no more than around 2 or 3 millimetres. This prevents the orthodontic screw 114′ from integrating with the osseous tissue. The two-part orthodontic screws 114 are inserted into the edentulous area of the maxillary or mandibular arch in the same manner.

There is also a process for creating a dental implant drill-guide to guide the drilling of dental-implant cavities in an edentulous area of a maxillary or mandibular arch is illustrated in FIG. 16, referenced globally at S200. The entire process can therefore be summarised as follows. A plurality of apical parts 38, 138, 138′ of two-part orthodontic screws 14, 114, 114′ can be inserted, at step S201, into the edentulous area of the maxillary or mandibular arch 10, 12 of a patient, such that the two-part orthodontic screws 14, 114, 114′ connect with the osseous tissue of the edentulous area of a maxillary or mandibular arch 10, 12. Impression data of the edentulous area of the maxillary or mandibular arch 10, 12 inclusive of the plurality of two-part orthodontic screws can then be obtained, at step S202, to form a maxillary or mandibular image. The impression data can then be refined, at step S203, based on characteristic shape data that corresponds to the two-part orthodontic screws 14, 114, 114′. The characteristic shape data of the two-part orthodontic screws 14, 114, 114′ is predetermined and relates to the screw selected for use by the dental practitioner.

Implant position data for the one or more dental implants can be determined, at step S204, from the refined impression for the insertion of dental implants 16 into the edentulous area of the maxillary or mandibular arch 10, 12 relative to the positions of the plurality of two-part orthodontic screws 14, 114, 114′. A dental implant drill-guide 24 can be created, at step S205, from the determined dental implant position data, such that it takes into account the one or more dental implant positions and the positions of the plurality of two-part orthodontic screws 14, 114, 114′. In this case, it is the apical head 42, 142, 142′ of the plurality of two-part orthodontic screws 14, 114, 114′ that shape-matingly engages with the plurality of orthodontic screw-seats 26.

By refining the impression data with predetermined characteristic shape of the two-part orthodontic screws 14, 114, 114′, it is possible to create a well-fitting dental implant drill-guide 24 as the orthodontic screws 14, 114, 114′, provide an anchor for the dental implant drill-guide 24 which is fixed relative to the osseous tissue 18 of the edentulous area of the maxillary or mandibular arch 10, 12. The refinement also allows for the precise determination of the dental implant position data for the insertion of one or more dental implants 16 relative to the positions of the plurality of orthodontic screws 14, 114, 114′. The process can be used with any two-part orthodontic screws, it is not specific to the two-part orthodontic screws described herein.

Whilst dental implants are suggested as being the desired items to install into the patient's mouth, it will be appreciated that drill-guide geometry formed in accordance with the present invention could be used in the context of any oral drilling application, either for edentulous patients or partially edentulous patients.

Whilst it is suggested that the same set of orthodontic screws may be used both to reference the position of the osseous tissue in the patient and then to secure the dental implant drill guide in position, it may be possible to use a first set of orthodontic screws in the referencing stage, and then only utilise the two-part screws at a later juncture.

It is therefore possible to provide a method of more accurately and reproducibly drilling dental implant cavities into the edentulous maxillary or mandibular arch of a patient by manufacturing a dental implant drill-guide which is bespoke to the patient. This can be achieved by imaging the edentulous maxillary or mandibular arch of the patient following the insertion of a plurality of orthodontic screws therein. This means that the images taken of the mandible or maxilla have a fixed reference thereon, and from the positions of the orthodontic screws, an appropriately-shaped drill guide can be produced which is seatable upon the orthodontic screws.

The words ‘comprises/comprising’ and the words ‘having/including’ when used herein with reference to the present invention are used to specify the presence of stated features, integers, steps or components, but do not preclude the presence or addition of one or more other features, integers, steps, components or groups thereof.

It is appreciated that certain features of the invention, which are, for clarity, described in the context of separate embodiments, may also be provided in combination in a single embodiment. Conversely, various features of the invention which are, for brevity, described in the context of a single embodiment, may also be provided separately or in any suitable sub-combination.

The embodiments described above are provided by way of examples only, and various other modifications will be apparent to persons skilled in the field without departing from the scope of the invention as defined herein.

1. A method of creating a dental implant drill-guide for guiding the drilling of dental-implant cavities in an edentulous maxillary or mandibular arch, the method comprising the steps of:
a] using mucosal impression data of an edentulous maxillary or mandibular arch having a plurality of orthodontic screws inserted into the osseous tissue thereof and which extend through the gingival mucosa, along with image data of the edentulous maxillary or mandibular arch to determine dental implant position data for the insertion of one or more dental implants into the edentulous maxillary or mandibular arch relative to the positions of the plurality of orthodontic screws;
b] forming dental implant drill-guide parameter data from the determined dental implant position data; and
c] creating a dental implant drill-guide from the determined dental implant drill-guide parameter data, the dental implant drill-guide having a plurality of orthodontic screw-seats which correspond to the said plurality of orthodontic screws of the mucosal impression data.

2. A method as claimed in claim 1, wherein during step a], the mucosal impression data is determined using three said orthodontic screws which are inserted in a triangular arrangement in the edentulous maxillary or mandibular arch.

3. A method as claimed in claim 1, wherein during step a], the mucosal impression data is determined using the plurality of orthodontic screws which are inserted between 2 and 3 millimetres deep into the osseous tissue of the edentulous maxillary or mandibular arch.

4. A method as claimed in claim 1, wherein during step a] a digital mucosal impression of the edentulous maxillary or mandibular arch is obtained.

5. A method as claimed in claim 1, further comprising a step prior to step a] of obtaining pre-operative imaging data of the edentulous maxillary or mandibular arch to determine an optimal position for the plurality of orthodontic screws and/or an optimal position of the dental-implant cavities.

6. A method as claimed in claim 5, wherein the said pre-operative imaging data is radiographic imaging data.

7. A method as claimed in claim 1, wherein during step a], the imaging data is Cone Beam Computed Tomography imaging data.

8. A method as claimed in claim 1, wherein during step c], further comprising the step of engaging the orthodontic screw seats of the dental implant with the plurality of orthodontic screws using a positive locator.

9. A method as claimed in claim 8, wherein the positive locators include a collar of each orthodontic screw which is receivable by the dental implant drill-guide.

10. A method as claimed in claim 1, wherein during step a], the mucosal impression data is refined using pre-existing characteristic shape data corresponding to the two-part orthodontic screws.

11. A method of creating a dental implant drill-guide for guiding the drilling of dental-implant cavities in an edentulous area of a maxillary or mandibular arch, the method comprising the steps of:
a] inserting a plurality of apical parts of two-part orthodontic screws into an edentulous area of a maxillary or mandibular arch of a patient, such that the two-part orthodontic screws engage with the osseous tissue of the edentulous area of the maxillary or mandibular arch and extend through the gingival mucosa;
b] obtaining impression data of the edentulous area of the maxillary or mandibular arch inclusive of the plurality of two-part orthodontic screws to form a maxillary or mandibular image;
c] refining the impression data based on characteristic shape data corresponding to the two-part orthodontic screws;
d] determining dental implant position data for the insertion of one or more dental implants into the edentulous area of the maxillary or mandibular arch relative to the positions of the plurality of two-part orthodontic screws from the refined impression data; and
e] creating a dental implant drill-guide from the determined dental implant position data, the dental implant drill-guide having a plurality of orthodontic screw-seats which correspond to the said plurality of two-part orthodontic screws.

12. A method as claimed in claim 11, wherein the coronal part of each of the two-part orthodontic screws, has a screw-thread which is receivable within an apical part of the two-part orthodontic screw.

13. A method as claimed in claim 11, wherein the characteristic shape data relates to a shape of the two-part orthodontic screw which is: pyramidal frustum; substantially pyramidal frustum; conical frustum; or substantially conical frustum.

14. A method as claimed in claim 11, wherein during step a], the two-part orthodontic screws are inserted in a triangular arrangement into the edentulous area of the maxillary or mandibular arch.

15. A method as claimed in claim 11, wherein during step a], the plurality of orthodontic screws is inserted between 2 and 3 millimetres deep into the osseous tissue of the edentulous area of the maxillary or mandibular arch.

16. A method as claimed in claim 11, wherein during step b], image data of the edentulous area of the maxillary or mandibular arch is used alongside the impression data to form the maxillary or mandibular image.

17. A method as claimed in claim 11, further comprising a step prior to step a] of obtaining pre-operative imaging data of the edentulous area of the maxillary or mandibular arch to determine an optimal position for the plurality of orthodontic screws and/or an optimal position of the dental-implant cavities.

18. A method as claimed in claim 11, wherein the said pre-operative imaging data is radiographic imaging data.

19. A method of implanting one or more dental implants into a patient, the method comprising the steps of:
a] inserting a plurality of apical parts of two-part orthodontic screws into an edentulous maxillary or mandibular arch of a patient, such that the two-part orthodontic screws engage with the osseous tissue of the edentulous maxillary or mandibular arch;
b] obtaining three-dimensional oral information data of the edentulous maxillary or mandibular arch;
c] imaging the maxilla or mandible of the patient inclusive of the plurality of two-part orthodontic screws to form a maxillary or mandibular image;
d] determining from the maxillary or mandibular image and the three-dimensional oral information data one or more dental implant positions for the insertion of dental implants into the edentulous maxillary or mandibular arch relative to the positions of the plurality of two-part orthodontic screws;
e] creating a dental implant drill-guide based on the one or more dental implant positions and the positions of the plurality of two-part orthodontic screws;
f] positioning the dental drill-guide on the apical parts of the two-part orthodontic screws;
g] fixing the dental implant drill-guide in position by engaging the coronal parts of the two-part orthodontic screws with the apical parts of the two-part orthodontic screws so as to clamp the dental implant drill-guide;
h] drilling one or more dental-implant cavities in the edentulous maxillary or mandibular arch using the dental implant drill-guide; and
i] inserting a dental implant into each dental-implant cavity.

20. A method as claimed in claim 19, wherein, during step i], the dental implants are guided into the respective dental-implant cavities by the dental implant drill-guide.
